(19) United States
(12) Patent Application Publication
(10) Pub. No.: US 2023/0173281 A1
(43) Pub. Date: Jun. 8, 2023

(54) HYPERTENSION HIS BUNDLE PACING

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 17/911,561

(22) PCT Filed: Mar. 23, 2021
(86) PCT No.: PCT/EP2021/057384

§ 371 Date: Sep. 14, 2022

(30) Foreign Application Priority Data
Mar. 27, 2020 (EP) 20166128.7

(51) Int. Cl. A61N 1/365

(52) U.S. Cl. CPC A61N 1/365; A61N 1/36564; A61N 1/3684

(57) ABSTRACT

The present invention relates to an implantable device for stimulating a heart and lowering blood pressure, comprising: a stimulation unit configured to stimulate the His bundle or the left-bundle branch of the heart, and a sensing unit for detecting atrial activity of the heart. According to the present invention the stimulation unit is configured to stimulate the His bundle or the left bundle branch immediately before or immediately after detection of an atrial activity.

### CROSS-REFERENCE TO RELATED APPLICATIONS

This application is the United States National Phase under 35 U.S.C. § 371 of PCT International Patent Application No. PCT/EP2021/057384, filed on Mar. 23, 2021, which claims the benefit of European Patent Application No. 20166128.7, filed on Mar. 27, 2020, the disclosures of which are hereby incorporated by reference herein in their entireties.

### TECHNICAL FIELD

The present invention relates to a device and a method for treating hypertension by means of cardiac stimulation therapy.

### BACKGROUND

A method for treating hypertension is, for example, disclosed in U.S. Pat. No. 9,333,352 which describes methods for blood pressure lowering heart stimulation. The methods are based on making the timing inefficient so that the cardiac output decreases and, as a result, the blood pressure. As an example, U.S. Pat. No. 9,333,352 teaches to deliver ventricular stimulation at the same time as atrial activity, or shortly after, or before. Particularly, U.S. Pat. No. 9,333,352 discloses to deliver ventricular stimulation with a time delay of 0 to 50 ms after atrial activity.

Thus, for patients with pacemakers, blood pressure can be lowered by physiologically inefficient stimulation in the atrium (e.g., two stimulations per cycle). However, for patients with intact AV transition this may mean non-essential right-sided stimulation. Furthermore, ventricular stimulation leads to a non-physiological spread of excitation and is therefore not recommended over a longer period of time.

Therefore, a problem to be solved by the present invention is to provide an implantable device and a method that allow reducing the blood pressure permanently in combination with a cardiac stimulation that reduces the risk of the above-described unwanted effect of unnecessary right ventricular stimulation.

At least this problem is solved by an implantable device having the features of claim 1 as well as by a method having the features of claim 9.

Preferred embodiments of these aspects of the present invention are stated in the corresponding sub-claims and are described below.

According to claim 1, an implantable device for stimulating a heart and lowering blood pressure of a patient is disclosed, comprising:
a stimulation unit configured to stimulate the His bundle or the left bundle branch of the heart of the patient, and
a sensing unit for detecting atrial activity of the heart.

According to the present invention, the stimulation unit is configured to stimulate the His bundle or the left bundle branch immediately before or immediately after detection of an atrial activity. Particularly, in an embodiment, the stimulation unit is configured to stimulate either the His bundle or the left bundle branch.

The His bundle is part of the electrical conduction system of the heart. It serves to transmit impulses from the atrioventricular node to the ventricles of the heart. The bundle of His branches into the left bundle branch and the right bundle branch, which run along the interventricular septum. In this regard the present invention is particularly based on the idea to deliver a His-Pace (virtually without transition time) immediately after (or before) atrial activity, in order to prematurely terminate a ventricular filling phase by atrial contraction and cause blood pressure reduction. After a predefined number of cycles, His-paces can be suspended for several cycles.

According to an embodiment, the atrial sensing unit is configured to detect atrial activity of the heart using a ground electrode contact located outside the heart. In an embodiment, the housing of the implantable device can be used as the ground electrode contact. In a further embodiment, the ground electrode contact can be part of the housing of the implantable device. In case atrial activity is detected using two electrode contacts placed in the atrium and one of these contacts being placed at the His bundle or very close to the His bundle, destruction can come from signals originating from the His bundle. If the ground electrode contact is located outside the heart, the detection is not destructed by cardiac signals.

According to an embodiment, the stimulation unit is configured to conduct His stimulation or left bundle branch stimulation immediately before or immediately after the perception of an atrial depolarization by the sensing unit without the use of an AV time.

Particularly, stimulating the His bundle or the left-bundle branch immediately before or immediately after detection of an atrial activity corresponds to stimulating the His bundle or the left-bundle branch by means of the stimulation unit less than 80 ms, preferably less than 70 ms, preferably less than 60 ms, preferably less than 50 ms, preferably less than 40 ms, preferably less than 30 ms, preferably less than 20 ms, preferably less than 10 ms before or after detection of an atrial activity.

According to a preferred embodiment of the implantable device, the implantable device/stimulation unit is designed to stimulate the His bundle or the left-bundle branch less than 80 ms, preferably less than 70 ms, preferably less than 60 ms, preferably less than 50 ms, preferably less than 40 ms, preferably less than 30 ms, preferably less than 20 ms, preferably less than 10 ms before detection of an atrial activity.

Furthermore, according to a preferred embodiment of the implantable device, the implantable device/stimulation unit is designed to stimulate the His bundle or the left-bundle branch less than 80 ms, preferably less than 70 ms, preferably less than 60 ms, preferably less than 50 ms, preferably less than 40 ms, preferably less than 30 ms, preferably less than 20 ms, preferably less than 10 ms after detection of an atrial activity.

Particularly, the phase of His stimulation and/or left bundle branch stimulation can be cyclically replaced by a phase without stimulation. Particularly, the implantable device may comprise a sequence control system that phasewise (cyclically) suspends the His bundle stimulation and/or left bundle branch stimulation for a predetermined time or number of heart cycles

According to an embodiment, the implantable device, particularly the stimulation unit, is configured to suspend the stimulation of the His bundle and/or of the left bundle branch.

According to a preferred embodiment of the present invention, the implantable device (particularly the stimulation unit) is configured to suspend the stimulation of the His bundle and/or the left bundle branch after a predefined number of heart cycles or after a predetermined time period for at least one heart cycle or for a predetermined time period, wherein preferably said number is a natural number between one and ten, i.e., the His bundle and/or the left bundle branch stimulation may be suspended for one of: 1 cycle, 2 cycles, 3 cycles, 4 cycles, 5 cycles, 6 cycles, 7 cycles, 8 cycles, 9 cycles, 10 cycles.

Furthermore, according to an embodiment, the implantable device, particularly the stimulation unit, is configured to control suspension of the stimulation of the His bundle and/or the left bundle branch depending on one or more parameters recorded by the implantable device or recorded by another device, which parameter(s) is/are indicative of systemic blood pressure.

According to yet a further embodiment of the present invention, the implantable device comprises an electrode lead electrically connected to the stimulation unit and the sensing unit, wherein the electrode lead comprises a first electrode contact configured to be fixed to a proximal portion of the His bundle, and a second electrode contact for sensing said atrial activity.

Furthermore, regarding left bundle branch stimulation, the implantable device may comprise an electrode lead comprising an electrode contact configured to be positioned in the ventricular, deep septal region of the left bundle branch.

A further aspect of the present invention relates to method for lowering blood pressure of a patient suffering from hypertension by means of His bundle or left bundle branch stimulation with electrical stimulation pulses, particularly using an implantable device according to the present invention, wherein the His bundle stimulation or the left bundle branch stimulation is applied to the heart immediately before or immediately after detection of an atrial activity of the heart. The features described above in conjunction with the implantable device apply for the method according to the present invention as well and may be used to further specify steps of the method according to the present invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following embodiments, further features and advantages of the present invention shall be described with reference to the Figures which depict preferred embodiments of the present invention, wherein:

FIG. 1 shows an embodiment of an implantable device according to the present invention;

FIG. 2 shows a flow chart, wherein the functions of an embodiment of the implantable device as well as of the method according to the present invention may be carried out according to this flow chart; and

FIG. 3 shows schematically an electrode lead according to an embodiment of the present invention for achieving stimulation of the left bundle branch (LBB).

### DETAILED DESCRIPTION

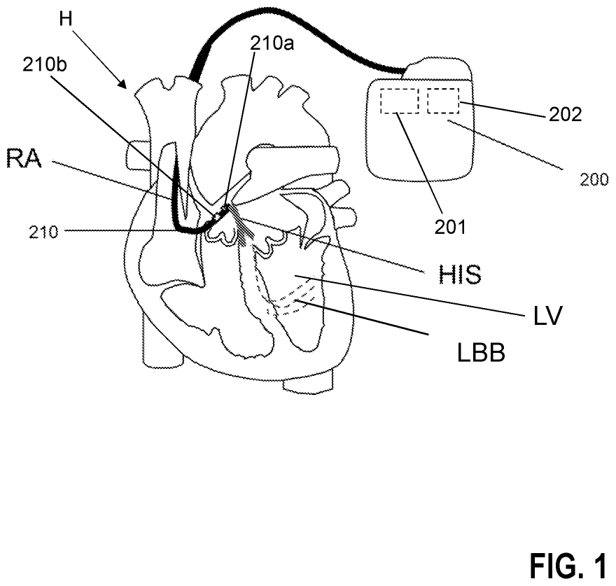

FIG. 1 shows an embodiment of an implantable medical device 200 according to the present invention for conducting a blood pressure-lowering stimulation of the heart H of a patient that suffers from hypertension. The device 200 comprises a stimulation unit 201 configured to stimulate the His bundle HIS or the left-bundle branch LBB of the heart H of the patient, and a sensing unit 202 for detecting atrial activity of the heart H. According to the present invention, the stimulation unit 201 is configured to stimulate the His bundle HIS or the left bundle branch LBB immediately before or immediately after detection of an atrial activity sensed by the sensing unit 202.

Furthermore, the device 200 preferably comprises an electrode lead 210 with at least two electrode contact 210a, 210b. The electrode lead 210 is configured to be implanted in such a way that a first electrode contact 210a forming a stimulation electrode contact is fixed in the proximal region of the His bundle HIS and at least a second electrode contact 210b is arranged in such a way that the intrinsic activation of the right atrium RA can be perceived via the sensing unit 202. To this end, the second electrode contact 210b is connected to the stimulation unit 201 and the first electrode contact 210a is connected to the stimulation unit 201 to apply electrical stimulation pulses to the His bundle HIS.

For stimulating the left bundle branch LBB, the device can comprise an electrode lead which is implanted in a ventricular, deep septal position to reach the LBB.

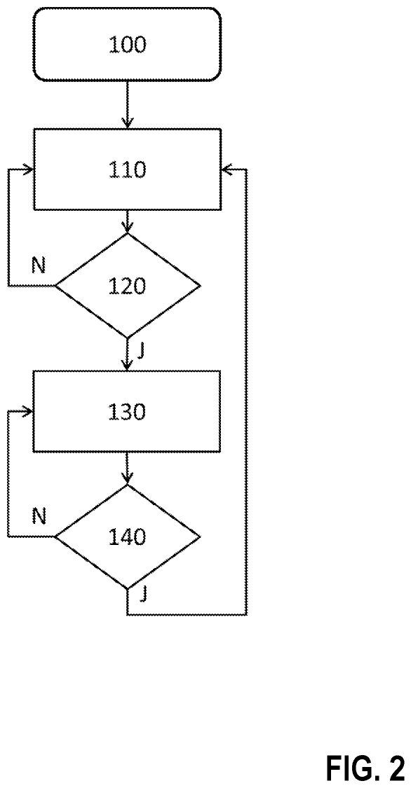

FIG. 2 shows a flow chart of a method for lowering blood pressure. Particularly, the implantable device 200 can be configured to perform these steps.

First, the blood pressure-lowering therapy is initiated 100 by programming and activating the stimulation parameters.

Subsequently, the blood pressure-lowering His bundle stimulation takes place by stimulating 110 the His bundle HIS immediately after each atrial perception, which causes the filling of the right ventricle to end prematurely (AV time shortened by the AV-His time) due to the atrial contraction (reduced preload) and as a result there is less wall tension in the right ventricle, which in turn causes a negative inotropy and thus a reduction in the blood pressure for this heartbeat. This stimulation is repeated as long as a predefined number of His stimulations 120, N have taken place.

When the number of stimulated His intervals is reached 120, J, the His stimulation is switched off 130 and the rhythm is only monitored.

His stimulation is stopped with the aim of preventing blood pressure counter regulation by the baroreceptors.

After a defined number of intrinsic heartbeats 140, J, His stimulation 110 is reactivated.

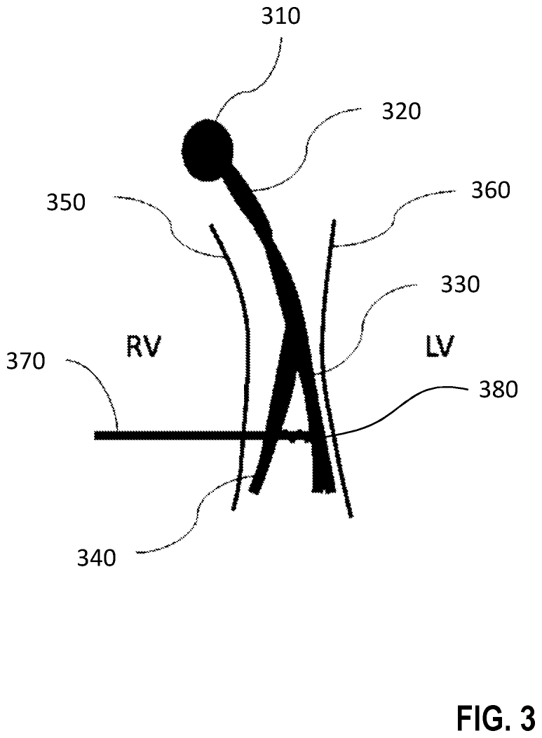

FIG. 3 schematically shows an electrode lead 370 according to an embodiment of the present invention in the implanted position, which can be screwed very deeply into the myocardium in order to reach the two left legs 330 of the LBB. The ventricular conduction system consists of the AV node 310, the HIS bundle 320, the two left legs 330 and the right leg 340. The electrode lead 370 is implanted from the right ventricle RV and positioned on the right ventricular septum 150. The stimulation pole 380 is positioned so deep in the septum that it is positioned in or near the left leg 330 and thus primarily stimulates the left leg before the division of the left leg.

The present invention advantageously enables a device-based therapy for permanent systemic blood pressure reduction with physiological stimulation.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable device for stimulating a heart and lowering blood pressure, comprising:
a stimulation unit configured to stimulate the His bundle or the left-bundle branch of the heart, and
a sensing unit for detecting atrial activity of the heart,
wherein
the stimulation unit is configured to stimulate the His bundle or the left bundle branch immediately before or immediately after detection of an atrial activity.

2. The implantable device according to claim 1, wherein the atrial sensing unit is configured to detect atrial activity of the heart using a ground electrode contact located outside the heart.

3. The implantable device according to claim 1, wherein the stimulation unit is configured to stimulate the His bundle or the left-bundle branch less than 80 ms before detection of an atrial activity.

4. The implantable device according to claim 1, wherein the stimulation unit is configured to stimulate the His bundle or the left-bundle branch less than 80 ms after detection of an atrial activity.

5. The implantable device according to claim 1, wherein the implantable device is configured to suspend the stimulation of the His bundle and/or of the left bundle branch.

6. The implantable device according to claim 1, wherein the implantable device is configured to suspend the stimulation of the His bundle and/or the left bundle branch after a predefined number of heart cycles or after a predetermined time period for at least one heart cycle or for a predetermined time period, wherein preferably said number is a natural number between one and ten.

7. The implantable device according to claim 1, wherein the implantable device is configured to control suspension of the stimulation of the His bundle and/or the left bundle branch depending on one or more parameters recorded by the implantable device or by another device and assignable to the systemic blood pressure.

8. The implantable device according to claim 1, wherein the implantable device comprises an electrode lead electrically connected to the stimulation unit and the sensing unit, wherein the electrode lead comprises a first electrode contact configured to be fixed to a proximal portion of the His bundle and to stimulate the His bundle, and a second electrode contact for sensing said atrial activity.

9. The implantable device according to claim 1, wherein the implantable device comprises an electrode lead comprising an electrode contact configured to be positioned in the region of the ventricular septum, wherein the implantable device is configured to stimulate the left bundle branch.

10. Method for lowering blood pressure by means of His bundle or left bundle branch stimulation using an implantable device according to claim 1, wherein His bundle stimulation or left bundle branch stimulation is applied to the heart immediately before or immediately after detection of an atrial activity of the heart.

11. The implantable device according to claim 1, wherein the stimulation unit is configured to stimulate the His bundle or the left-bundle branch less than 10 ms before detection of an atrial activity.

12. The implantable device according to claim 1, wherein the stimulation unit is configured to stimulate the His bundle or the left-bundle branch less than 10 ms after detection of an atrial activity.
